(19) United States
(12) Patent Application Publication
(10) Pub. No.: US 2018/0017580 A1
(43) Pub. Date: Jan. 18, 2018

(54) BIOMARKERS FOR DISTINGUISHING MOOD DISORDERS

(71) Applicant: Myriad Genetics, Inc., Salt Lake City, UT (US)

(72) Inventor: Rejesh Kaldate, Salt Lake City, UT (US)

(21) Appl. No.: 15/614,375

(22) Filed: Jun. 5, 2017

Related U.S. Application Data
(63) Continuation of application No. PCT/US15/63952, filed on Dec. 4, 2015.
(60) Provisional application No. 62/088,188, filed on Dec. 5, 2014.
(60) Provisional application No. 62/106,863, filed on Jan. 23, 2015.

(51) Int. Cl. G01N 33/68

(52) U.S. Cl. CPC G01N 33/6896; G01N 2800/60; G01N 2800/304; G01N 2800/52

(57) ABSTRACT

The invention relates to biomarkers useful for distinguishing Bipolar Disorder 1 (BPD1), Bipolar Disorder 2 (BPD2), and Major Depressive Disorder (MDD) and to methods for use of said biomarkers in differential diagnosis, treatment, altering treatment and generating quantitative data.

### RELATED APPLICATIONS

This application is a continuation of PCT Application No. PCT/US15/063952 (filed Dec. 4, 2015), and further claims the priority benefit of U.S. Provisional Application Ser. No. 62/106,863 (filed Jan. 23, 2015), and 62/088,118 (filed Dec. 5, 2014), which are each hereby incorporated by reference in their entirety.

### FIELD OF THE INVENTION

The invention relates to methods for distinguishing mood disorders.

### BACKGROUND OF THE INVENTION

Mood disorder diagnoses have traditionally been based on behavioral observation and self-reporting. Additionally, many individuals with bipolar disorder I or II will often originally present to a healthcare provider with depressive symptoms, as opposed to manic or hypomanic symptoms. Despite often presenting with depressive symptoms, it is of particular importance that individuals with BPD1 and BPD2 are not treated with antidepressants used to treat depression, as this can exacerbate manic or hypomanic episodes. Because of the complexities associated with early onset mood disorders, there is a need for biomarkers or proteomic signatures that will enhance the initial assessment, differential diagnosis, diagnostic confirmation, and treatment in major depression and bipolar disorder.

### BRIEF SUMMARY OF THE INVENTION

The following numbered list of embodiments briefly summarized many aspects of the present disclosure.

1. A method of altering treatment, comprising:

a. assaying a body fluid sample, from an individual presenting with depressive symptoms, to determine the amount in the sample of each of a panel of biomarkers;

b. determining, by a model comprising the amounts of each of the panel of biomarkers, that the individual likely has a mood disorder other than Major Depressive Disorder (MDD); and

c. altering the treatment of the individual from antidepressant therapy to treatment of the bipolar disorder.

2. The method of embodiment 1, wherein the individual is being treated with antidepressant therapy before the step of altering the treatment of the individual.

3. The method of embodiment 2, wherein the antidepressant therapy comprises treating the individual with a drug-based or non-drug-based antidepressant therapy, or both.

4. The method of embodiment 3, wherein the drug-based antidepressant therapy comprises a drug selected from a selective serotonin reuptake inhibitor (SSRI), serotonin-norepinephrine reuptake inhibitors (SNRI), norepinephrine and dopamine reuptake inhibitor (NDRI), tetracyclic antidepressant (tetracyclic), tricyclic antidepressant (tricyclic) or monoamine oxidase inhibitor (MAOI), or combinations thereof.

5. The method of embodiment 3, wherein the non-drug-based antidepressant therapy comprises a treatment selected from cognitive-behavioral therapy, psychotherapy, psychodynamic therapy, electroconvulsive therapy, hospitalization and residential treatment programs, vagus nerve stimulation, transcranial magnetic stimulation or regular, vigorous exercise or combinations thereof.

6. The method of any of embodiments 1-5, wherein the bipolar disorder is Bipolar Disorder I (BPD1).

7. The method of any of embodiments 1-5, wherein the bipolar disorder is Bipolar Disorder II (BPD2).

8. The method of any of embodiments 1-7, wherein the body fluid sample comprises urine, blood, plasma, serum, buffy coat, or saliva.

9. The method of embodiment 8, wherein the body fluid sample comprises blood, plasma or serum.

10. The method of any of embodiments 1-9, wherein the model is generated using a statistical method comprising Multivariate Logistic Regression (MLR), Principal Component Analysis (PCA), Factor Analysis, Partial Least Squares Regression (PLS), Neural Network, Linear Discriminant Analysis (LDA), K-Nearest Neighbor (KNN) classification, Cluster Analysis or self-organizing maps, or combinations thereof.

11. The method of embodiment 10, wherein the model is generated using LDA or MLR.

12. The method of any of embodiments 1-11, wherein assaying the body fluid sample comprises using Western Blot, Enzyme-Linked Immunosorbent Assay (ELISA), liquid chromatography mass spectrometry (LCMS), surface enhanced laser desorption ionization time of flight mass spectrometry (SELDI-TOF), two-dimension gel electrophoresis (2-DE), and/or any combinations thereof.

13. The method of any of embodiments 1-11, wherein assaying the body fluid sample comprises using multiplexed immune assays.

14. The method of embodiment 13, wherein the multiplexed immune assay comprises a microsphere-based assay.

15. The method of any of embodiments 13-14, wherein between 1 and 15 multiplexed assays are performed in a single reaction vessel.

16. The method of embodiment 14, wherein the multiplexed assay includes analyte-specific microspheres labeled with a unique fluorescent signature.

17. The method of any of embodiments 13-16, wherein the multiplexed assay includes an assay-specific capture reagent that is covalently conjugated to a set of unique microspheres.

18. The method of embodiment 17, wherein the assay-specific capture reagent is an antibody.

19. The method of embodiment 17, wherein a detecting reagent is added to the assay.

20. The method of embodiment 19, wherein the detecting reagent is an antibody, antigen or ligand.

21. The method of any of embodiments 19-20, wherein the assay kinetics are near solution-phase.

22. The method of any of embodiments 1-21, wherein the multiplex reaction is passed through an analyzer comprising laser beams.

23. The method of embodiment 22, wherein the analyzer uses hydrodynamic focusing to pass microspheres through the analyzer in single file.

24. The method of any of embodiments 1-23, wherein an encoded fluorescent signature of a microsphere is analyzed.

25. The method of any of embodiments 1-24, wherein the median value of the analyte-specific fluorescence is measured.

26. The method of embodiment 25, wherein the analyte-specific fluorescence is calibrated using internal controls of known quantity.

27. The method of any of embodiments 1-26, wherein altering the treatment of the individual comprises treating the individual with lithium.

28. The method of any of embodiments 1-26, wherein altering the treatment of the individual comprises treating the individual with an anticonvulsant.

29. The method of embodiment 28, wherein the anticonvulsant is valproate, lamotrigine or carbamazepine.

30. The method of any of embodiments 1-26, wherein altering the treatment of the individual comprises treating the individual with a traditional antipsychotic.

31. The method of embodiment 30, wherein the traditional antipsychotic is haloperidol, loxapine or chlorpromazine.

32. The method of any of embodiments 1-26, wherein altering the treatment of the individual comprises treating the individual with an atypical antipsychotic.

33. The method of embodiment 32, wherein, wherein the atypical antipsychotic is aripiprazole, risperidone, asenapine, quetiapine, ziprasidone, olanzapine or lurasidone.

34. The method of any of embodiments 1-26, wherein altering the treatment of the individual comprises treating the individual with a benzodiazepine.

35. The method of embodiment 34, wherein, wherein the benzodiazepine is alprazolam, lorazepam or diazepam.

36. The method of any of embodiments 1-26, wherein altering the treatment of the individual comprises treating electroconvulsive therapy (ECT).

37. The method of any of embodiments 1-36, wherein the panel of biomarkers comprises the proteins listed in Table 1, 2, 3, 4, 5, 6, 7, 8, 9, 10, 11, 12, 13 or 14.

38. The method of any of embodiments 1-36, wherein the panel of biomarkers comprises a core group of three proteins listed in Table 15 or 16.

39. The method of any of embodiments 1-36, wherein the panel of biomarkers comprises a sub-panel of three proteins listed in Table 17.

40. A method of treatment, comprising:

a. assaying a body fluid sample, from an individual presenting with depressive symptoms, to determine the amount in the sample of each of a panel of biomarkers;

b. determining, by a model comprising the amounts of each of the panel of biomarkers, that the individual likely has a mood disorder other than Major Depressive Disorder (MDD); and

c. administering a treatment to the individual which alleviates the symptoms of the bipolar disorder.

41. The method of embodiment 40, wherein the bipolar disorder is Bipolar Disorder I (BPD1).

42. The method of embodiment 40, wherein the bipolar disorder is Bipolar Disorder II (BPD2).

43. The method of any of embodiments 40-42, wherein the body fluid sample comprises urine, blood, plasma, serum, buffy coat, or saliva.

44. The method of embodiment 43, wherein the body fluid sample comprises blood, plasma or serum.

45. The method of any of embodiments 40-44, wherein the model is generated using a statistical method comprising Multivariate Logistic Regression (MLR), Principal Component Analysis (PCA), Factor Analysis, Partial Least Squares Regression (PLS), Neural Network, Linear Discriminant Analysis (LDA), K-Nearest Neighbor (KNN) classification, Cluster Analysis or self-organizing maps, or combinations thereof.

46. The method of embodiment 45, wherein the model is generated using LDA or MLR.

47. The method of any of embodiments 40-46, wherein assaying the body fluid sample comprises using Western Blot, Enzyme-Linked Immunosorbent Assay (ELISA), liquid chromatography mass spectrometry (LCMS), surface enhanced laser desorption ionization time of flight mass spectrometry (SELDI-TOF), two-dimension gel electrophoresis (2-DE), and/or any combinations thereof.

48. The method of any of embodiments 40-46, wherein assaying the body fluid sample comprises using multiplexed immune assays.

49. The method of embodiment 48, wherein the multiplexed immune assay comprises a microsphere-based assay.

50. The method of any of embodiments 48-49, wherein between 1 and 15 multiplexed assays are performed in a single reaction vessel.

51. The method of embodiment 49, wherein the multiplexed assay includes analyte-specific microspheres labeled with a unique fluorescent signature.

52. The method of any of embodiments 48-51, wherein the multiplexed assay includes an assay-specific capture reagent that is covalently conjugated to a set of unique microspheres.

53. The method of embodiment 52, wherein the assay-specific capture reagent is an antibody.

54. The method of embodiment 52, wherein a detecting reagent is added to the assay.

55. The method of embodiment 54, wherein the detecting reagent is an antibody, antigen or ligand.

56. The method of any of embodiments 48-55, wherein the assay kinetics are near solution-phase.

57. The method of any of embodiments 40-56, wherein the multiplex reaction is passed through an analyzer comprising laser beams.

58. The method of embodiment 57, wherein the analyzer uses hydrodynamic focusing to pass microspheres through the analyzer in single file.

59. The method of any of embodiments 40-58, wherein an encoded fluorescent signature of a microsphere is analyzed.

60. The method of any of embodiments 40-59, wherein the median value of the analyte-specific fluorescence is measured.

61. The method of embodiment 60, wherein the analyte-specific fluorescence is calibrated using internal controls of known quantity.

62. The method of any of embodiments 40-61, wherein altering the treatment of the individual comprises treating the individual with lithium.

63. The method of any of embodiments 40-61, wherein altering the treatment of the individual comprises treating the individual with an anticonvulsant.

64. The method of embodiment 63, wherein the anticonvulsant is valproate, lamotrigine or carbamazepine.

65. The method of any of embodiments 40-61, wherein altering the treatment of the individual comprises treating the individual with a traditional antipsychotic.

66. The method of embodiment 65, wherein the traditional antipsychotic is haloperidol, loxapine or chlorpromazine.

67. The method of any of embodiments 40-61, wherein altering the treatment of the individual comprises treating the individual with an atypical antipsychotic.

68. The method of embodiment 67, wherein, wherein the atypical antipsychotic is aripiprazole, risperidone, asenapine, quetiapine, ziprasidone, olanzapine or lurasidone.

69. The method of any of embodiments 40-61, wherein altering the treatment of the individual comprises treating the individual with a benzodiazepine.

70. The method of embodiment 69, wherein, wherein the benzodiazepine is alprazolam, lorazepam or diazepam.

71. The method of any of embodiments 40-61, wherein altering the treatment of the individual comprises treating electroconvulsive therapy (ECT).

72. The method of any of embodiments 40-71, wherein the panel of biomarkers comprises the proteins listed in Table 1, 2, 3, 4, 5, 6, 7, 8, 9, 10, 11, 12, 13 or 14.

73. The method of any of embodiments 40-71, wherein the panel of biomarkers comprises a core group of three proteins listed in Table 15 or 16.

74. The method of any of embodiments 40-71, wherein the panel of biomarkers comprises a sub-panel of three proteins listed in Table 17.

75. A method for generating quantitative data for a subject comprising:

performing at least one immunoassay on a body fluid sample from the subject to generate a dataset comprising the quantitative data, wherein the quantitative data represents a panel of protein biomarkers, and wherein the individual has a mood disorder other than Major Depressive Disorder (MDD).

76. The method of embodiment 75, wherein performance of the at least one immunoassay comprises:

a. obtaining the body fluid sample, wherein the body fluid sample comprises the panel of protein biomarkers;

b. contacting the first sample with a plurality of distinct reagents;

c. generating a plurality of distinct complexes between the reagents and markers; and

d. detecting the complexes to generate the data.

77. The method of embodiment 75, wherein the bipolar disorder is Bipolar Disorder I (BPD1).

78. The method of embodiment 75, wherein the bipolar disorder is Bipolar Disorder II (BPD2).

79. The method of any of embodiments 75-78, wherein the body fluid sample comprises urine, blood, plasma, serum, buffy coat, or saliva.

80. The method of embodiment 79, wherein the body fluid sample comprises blood, plasma or serum.

81. The method of any of embodiments 75-80, wherein performance of the at least one immunoassay comprises performing Western Blot or Enzyme-Linked Immunosorbent Assay (ELISA).

82. The method of any of embodiments 75-80, wherein performance of the at least one immunoassay comprises performing multiplexed immune assays.

83. The method of embodiment 82, wherein the multiplexed immune assay comprises a microsphere-based assay.

84. The method of any of embodiments 82-83, wherein between 1 and 15 multiplexed assays are performed in a single reaction vessel.

85. The method of embodiment 83, wherein the multiplexed assay includes analyte-specific microspheres labeled with a unique fluorescent signature.

86. The method of any of embodiments 82-85, wherein the multiplexed assay includes an assay-specific capture reagent that is covalently conjugated to a set of unique microspheres.

87. The method of embodiment 86, wherein the assay-specific capture reagent is an antibody.

88. The method of embodiment 86, wherein a detecting reagent is added to the assay.

89. The method of embodiment 88, wherein the detecting reagent is an antibody, antigen or ligand.

90. The method of any of embodiments 82-89, wherein the assay kinetics are near solution-phase.

91. The method of any of embodiments 75-90, wherein the multiplex reaction is passed through an analyzer comprising laser beams.

92. The method of embodiment 91, wherein the analyzer uses hydrodynamic focusing to pass microspheres through the analyzer in single file.

93. The method of any of embodiments 75-92, wherein an encoded fluorescent signature of a microsphere is analyzed.

94. The method of any of embodiments 75-93, wherein the median value of the analyte-specific fluorescence is measured.

95. The method of embodiment 94, wherein the analyte-specific fluorescence is calibrated using internal controls of known quantity.

96. The method of any of embodiments 75-95, wherein the panel of biomarkers comprises the proteins listed in Table 1, 2, 3, 4, 5, 6, 7, 8, 9, 10, 11, 12, 13 or 14.

97. The method of any of embodiments 75-95, wherein the panel of biomarkers comprises a core group of three proteins listed in Table 15 or 16.

98. The method of any of embodiments 75-95, wherein the panel of biomarkers comprises a sub-panel of three proteins listed in Table 17.

99. A method of differential diagnosis, comprising:

a. obtaining a body fluid sample from an individual presenting with depressive symptoms;

b. assaying the body fluid sample to determine the amount in the sample of each of a panel of biomarkers;

c. determining, by a model comprising the amounts of each of the panel of biomarkers, that the individual likely has a mood disorder other than Major Depressive Disorder (MDD); and

d. diagnosing the individual as suffering from a bipolar disorder based at least in part on the determining in step b.

100. The method of embodiment 99, wherein the bipolar disorder is Bipolar Disorder I (BPD1).

101. The method of embodiment 99, wherein the bipolar disorder is Bipolar Disorder II (BPD2).

102. The method of any of embodiments 99-101, wherein the body fluid sample comprises urine, blood, plasma, serum, buffy coat, or saliva.

103. The method of embodiment 102, wherein the body fluid sample comprises blood, plasma or serum.

104. The method of any of embodiments 99-103, wherein assaying the body fluid sample performing Western Blot or Enzyme-Linked Immunosorbent Assay (ELISA).

105. The method of any of embodiments 99-103, wherein assaying the body fluid sample comprises performing multiplexed immune assays.

106. The method of embodiment 105, wherein the multiplexed immune assay comprises a microsphere-based assay.

107. The method of any of embodiments 105-106, wherein between 1 and 15 multiplexed assays are performed in a single reaction vessel.

108. The method of embodiment 106, wherein the multiplexed assay includes analyte-specific microspheres labeled with a unique fluorescent signature.

109. The method of any of embodiments 105-108, wherein the multiplexed assay includes an assay-specific capture reagent that is covalently conjugated to a set of unique microspheres.

110. The method of embodiment 109, wherein the assay-specific capture reagent is an antibody.

111. The method of embodiment 109, wherein a detecting reagent is added to the assay.

112. The method of embodiment 111, wherein the detecting reagent is an antibody, antigen or ligand.

113. The method of any of embodiments 105-112, wherein the assay kinetics are near solution-phase.

114. The method of any of embodiments 99-113, wherein the multiplex reaction is passed through an analyzer comprising laser beams.

115. The method of embodiment 114, wherein the analyzer uses hydrodynamic focusing to pass microspheres through the analyzer in single file.

116. The method of any of embodiments 99-115, wherein an encoded fluorescent signature of a microsphere is analyzed.

117. The method of any of embodiments 99-116, wherein the median value of the analyte-specific fluorescence is measured.

118. The method of embodiment 117, wherein the analyte-specific fluorescence is calibrated using internal controls of known quantity.

119. The method of any of embodiments 99-118, wherein the panel of biomarkers comprises the proteins listed in Table 1, 2, 3, 4, 5, 6, 7, 8, 9, 10, 11, 12, 13 or 14.

120. The method of any of embodiments 99-118, wherein the panel of biomarkers comprises a core group of three proteins listed in Table 15 or 16.

121. The method of any of embodiments 99-118, wherein the panel of biomarkers comprises a sub-panel of three proteins listed in Table 17.

122. A method of mitigating antidepressant-exacerbated mania or hypomania, comprising

a. assaying a body fluid sample, from an individual presenting with depressive symptoms, to determine the amount in the sample of each of a panel of biomarkers;

b. determining, by a model comprising the amounts of each of the panel of biomarkers, that the individual likely has a mood disorder other than Major Depressive Disorder (MDD); and

c. altering the treatment of the individual by: not beginning treatment with antidepressant therapy or discontinuing treatment with antidepressant therapy.

123. The method of embodiment 122, wherein the individual is being treated with antidepressant therapy before the step of altering the treatment of the individual.

124. The method of embodiment 123, wherein the antidepressant therapy comprises treating the individual with a drug-based therapy.

125. The method of embodiment 124, wherein the drug-based antidepressant therapy comprises a drug selected from a selective serotonin reuptake inhibitor (SSRI), serotonin-norepinephrine reuptake inhibitors (SNRI), norepinephrine and dopamine reuptake inhibitor (NDRI), tetracyclic antidepressant (tetracyclic), tricyclic antidepressant (tricyclic) or monoamine oxidase inhibitor (MAOI), or combinations thereof.

126. The method of any of embodiments 122-125, wherein the mood disorder other than MDD is Bipolar Disorder I (BPD1).

127. The method of any of embodiments 122-125, wherein the mood disorder other than MDD is Bipolar Disorder II (BPD2).

128. The method of any of embodiments 122-127, wherein the body fluid sample comprises urine, blood, plasma, serum, buffy coat, or saliva.

129. The method of embodiment 128, wherein the body fluid sample comprises blood, plasma or serum.

130. The method of any of embodiments 122-129, wherein the model is generated using a statistical method comprising Multivariate Logistic Regression (MLR), Principal Component Analysis (PCA), Factor Analysis, Partial Least Squares Regression (PLS), Neural Network, Linear Discriminant Analysis (LDA), K-Nearest Neighbor (KNN) classification, Cluster Analysis or self-organizing maps, or combinations thereof.

131. The method of embodiment 130, wherein the model is generated using LDA or MLR.

132. The method of any of embodiments 122-131, wherein assaying the body fluid sample comprises using Western Blot, Enzyme-Linked Immunosorbent Assay (ELISA), liquid chromatography mass spectrometry (LCMS), surface enhanced laser desorption ionization time of flight mass spectrometry (SELDI-TOF), two-dimension gel electrophoresis (2-DE), and/or any combinations thereof.

133. The method of any of embodiments 122-131, wherein assaying the body fluid sample comprises using multiplexed immune assays.

134. The method of embodiment 133, wherein the multiplexed immune assay comprises a microsphere-based assay.

135. The method of any of embodiments 133-134, wherein between 1 and 15 multiplexed assays are performed in a single reaction vessel.

136. The method of embodiment 134, wherein the multiplexed assay includes analyte-specific microspheres labeled with a unique fluorescent signature.

137. The method of any of embodiments 133-136, wherein the multiplexed assay includes an assay-specific capture reagent that is covalently conjugated to a set of unique microspheres.

138. The method of embodiment 137, wherein the assay-specific capture reagent is an antibody.

139. The method of embodiment 137, wherein a detecting reagent is added to the assay.

140. The method of embodiment 139, wherein the detecting reagent is an antibody, antigen or ligand.

141. The method of any of embodiments 139-140, wherein the assay kinetics are near solution-phase.

142. The method of any of embodiments 122-141, wherein the multiplex reaction is passed through an analyzer comprising laser beams.

143. The method of embodiment 142, wherein the analyzer uses hydrodynamic focusing to pass microspheres through the analyzer in single file.

144. The method of any of embodiments 122-143, wherein an encoded fluorescent signature of a microsphere is analyzed.

145. The method of any of embodiments 122-144, wherein the median value of the analyte-specific fluorescence is measured.

146. The method of embodiment 145, wherein the analyte-specific fluorescence is calibrated using internal controls of known quantity.

147. The method of any of embodiments 122-146, wherein altering the treatment of the individual comprises treating the individual with lithium.

148. The method of any of embodiments 122-146, wherein altering the treatment of the individual comprises treating the individual with an anticonvulsant.

149. The method of embodiment 148, wherein the anticonvulsant is valproate, lamotrigine or carbamazepine.

150. The method of any of embodiments 122-146, wherein altering the treatment of the individual comprises treating the individual with a traditional antipsychotic.

151. The method of embodiment 150, wherein the traditional antipsychotic is haloperidol, loxapine or chlorpromazine.

152. The method of any of embodiments 122-146, wherein altering the treatment of the individual comprises treating the individual with an atypical antipsychotic.

153. The method of embodiment 152, wherein, wherein the atypical antipsychotic is aripiprazole, risperidone, asenapine, quetiapine, ziprasidone, olanzapine or lurasidone.

154. The method of any of embodiments 122-146, wherein altering the treatment of the individual comprises treating the individual with a benzodiazepine.

155. The method of embodiment 154, wherein, wherein the benzodiazepine is alprazolam, lorazepam or diazepam.

156. The method of any of embodiments 122-146, wherein altering the treatment of the individual comprises treating electroconvulsive therapy (ECT).

157. The method of any of embodiments 122-156, wherein the panel of biomarkers comprises the proteins listed in Table 1, 2, 3, 4, 5, 6, 7, 8, 9, 10, 11, 12, 13 or 14.

158. The method of any of embodiments 122-156, wherein the panel of biomarkers comprises a core group of three proteins listed in Table 15 or 16.

159. The method of any of embodiments 122-156, wherein the panel of biomarkers comprises a sub-panel of three proteins listed in Table 17.

### DETAILED DESCRIPTION OF THE INVENTION

The invention relates to biomarkers useful for distinguishing Bipolar Disorder 1 (BPD1), Bipolar Disorder 2 (BPD2), and Major Depressive Disorder (MDD) and to methods for use of said biomarkers in differential diagnosis, treatment, altering treatment and generating quantitative data. The following detailed description provides details of many aspects and embodiments of the invention.

### Panels of Biomarkers for Distinguishing Mood Disorders

In some embodiments panels of biomarkers are utilized to distinguish between various mood disorders. In some embodiments, the panel of biomarkers comprises the proteins in Table 1. In some embodiments, the panel of biomarkers comprises the proteins in Table 2. In some embodiments, the panel of biomarkers comprises the proteins in Table 3. In some embodiments, the panel of biomarkers comprises the proteins in Table 4. In some embodiments, the panel of biomarkers comprises the proteins in Table 5. In some embodiments, the panel of biomarkers comprises the proteins in Table 6. In some embodiments, the panel of biomarkers comprises the proteins in Table 7. In some embodiments, the panel of biomarkers comprises the proteins in Table 8. In some embodiments, the panel of biomarkers comprises the proteins in Table 9. In some embodiments, the panel of biomarkers comprises the proteins in Table 10. In some embodiments, the panel of biomarkers comprises the proteins in Table 11. In some embodiments, the panel of biomarkers comprises the proteins in Table 12. In some embodiments, the panel of biomarkers comprises the proteins in Table 13. In some embodiments, the panel of biomarkers comprises the proteins in Table 14.

In some embodiments a panel may be comprised of a core group of biomarkers which provide a significant portion of the power of that particular panel of biomarkers in a particular model to distinguish between mood disorders.

In some embodiments, the core group of biomarkers provides a majority of the sensitivity of a model comprising the core group. In related embodiments, the core group provides at least 50.1% of the sensitivity of the model. In related embodiments, the core group provides at least 60% of the sensitivity of the model. In related embodiments, the core group provides at least 70% of the sensitivity of the model. In related embodiments, the core group provides at least 80% of the sensitivity of the model. In related embodiments, the core group provides at least 90% of the sensitivity of the model.

In some embodiments, the core group of biomarkers provides a majority of the specificity of a model comprising the core group. In related embodiments, the core group provides at least 50.1% of the specificity of the model. In related embodiments, the core group provides at least 60% of the specificity of the model. In related embodiments, the core group provides at least 70% of the specificity of the model. In related embodiments, the core group provides at least 80% of the specificity of the model. In related embodiments, the core group provides at least 90% of the specificity of the model.

In some embodiments, the core group of a panel of biomarkers comprises RBP 4, EGF and PARC.

In some embodiments, the core group of a panel of biomarkers comprises RBP 4, EGF and MMP 7

In some embodiments, the core group of a panel of biomarkers comprises RBP 4, PARC and MMP 7

In some embodiments, the core group of a panel of biomarkers comprises RBP 4, MMP 7 and TIMP 2

In some embodiments, the core group of a panel of biomarkers comprises EGF, PARC and MMP 7

In some embodiments, the core group of a panel of biomarkers comprises TTR, EGF and PARC

In some embodiments, the core group of a panel of biomarkers comprises TTR, EGF and MMP 7

In some embodiments, the core group of a panel of biomarkers comprises TTR, PARC and MMP 7

In some embodiments, the core group of a panel of biomarkers comprises TTR, MMP 7 and TIMP 2

In some embodiments, the core group of a panel of biomarkers comprises RBP 4, MMP 7 and MMP 2.

In some embodiments a panel may be comprised of a subpanel of biomarkers which provide a significant portion of the power of that particular panel of biomarkers in a particular model to distinguish between mood disorders.

In some embodiments, the subpanel of biomarkers provides a significant portion of the sensitivity of a model comprising the subpanel. In related embodiments, the core group provides at least 30% of the sensitivity of the model. In related embodiments, the core group provides at least 40% of the sensitivity of the model. In related embodiments, the core group provides at least 50% of the sensitivity of the model. In related embodiments, the core group provides at least 60% of the sensitivity of the model. In related embodiments, the core group provides at least 70% of the sensitivity of the model.

In some embodiments, the core group of biomarkers provides a significant portion of the specificity of a model comprising the core group. In related embodiments, the core group provides at least 30% of the specificity of the model. In related embodiments, the core group provides at least 40% of the specificity of the model. In related embodiments, the core group provides at least 50% of the specificity of the model. In related embodiments, the core group provides at least 60% of the specificity of the model. In related embodiments, the core group provides at least 70% of the specificity of the model.

In some embodiments, the subpanel of a panel of biomarkers comprises TTR, PARC and MMP 7.

In some embodiments, the subpanel of a panel of biomarkers comprises TTR, MMP 7 and TIMP 2.

In some embodiments, the subpanel of a panel of biomarkers comprises TTR, MMP 7 and MMP 2.

In some embodiments, the subpanel of a panel of biomarkers comprises TTR, MMP 7 and MMP 2.

In some embodiments, a panel of biomarkers comprises covariates. Covariate, as used in this application is defined as follows: given a first model comprising a first panel of biomarkers, said first panel of biomarkers comprising a first biomarker, a second biomarker is considered a covariate of the first biomarker if a second model comprising a second panel of biomarkers has an AUROC which is substantially similar to the first model, wherein the second panel of biomarkers is identical to the first panel of biomarkers except for the substitution of the first biomarker for the second biomarker.

In some embodiments, a covariate is substituted for a biomarker in a panel. In another embodiment, a covariate is added to a panel of biomarkers to complement a biomarker in a model. In some embodiments, a panel of biomarkers comprises one or more covariates from Tables 4, 6, 8, or 10. In a related embodiment, the panel of biomarkers comprises Hemopexin and TTR as covariates. In a related embodiment, the panel of biomarkers comprises Hemopexin and RBP4 as covariates. In a related embodiment, the panel of biomarkers comprises RBP4 and TTR as covariates. In a related embodiment, the panel of biomarkers comprises RBP4, Hemopexin and TTR as covariates. In a related embodiment, the panel of biomarkers comprises TAF1 and C3 as covariates. In a related embodiment, the panel of biomarkers comprises TIMP2 and MMP2 as covariates. In a related embodiment, the panel of biomarkers comprises Hemopexin and C3 as covariates. In a related embodiment, the panel of biomarkers comprises RBP4 and C3 as covariates. In a related embodiment, the panel of biomarkers comprises RBP4, Hemopexin and C3 as covariates. In a related embodiment, the panel of biomarkers comprises Hemopexin and SAP as covariates. In a related embodiment, the panel of biomarkers comprises C3 and SAP as covariates. In a related embodiment, the panel of biomarkers comprises C3, Hemopexin and SAP as covariates.

### Samples

In some embodiments a biological fluid sample is taken from a patient or individual. In some embodiments, the biological fluid sample is blood, or derived from blood. In related embodiments, the blood is obtained through venipuncture, finger stick or heel stick.

In some embodiments, the blood is collected in a container with added reagents. In related embodiments, the added reagents comprise an anti-coagulant.

In some embodiments the biological fluid sample is blood. In some embodiments the biological fluid sample from is plasma. In some embodiments the biological fluid sample from the patient may include urine, blood, plasma, serum, buffy coat or saliva.

In some embodiments, the biological fluid sample is serum, plasma or blood.

In some embodiments, the biological fluid sample is stored and/or shipped at room temperature with or without stabilizers. In some embodiments, the biological fluid sample is stored and/or shipped frozen. In some embodiments, the biological fluid sample is stored and/or shipped chilled. In some embodiments, serum, plasma or whole blood are dried on a material for storage and or shipping. In a related embodiment, the biological fluid sample is eluted from the storage material when ready to be tested at the testing site.

In some embodiments, the biological fluid sample is processed before it is assayed as described elsewhere herein. In one embodiment, the biological fluid sample is centrifuged. In a related embodiment, the biological fluid sample is blood, and after centrifugation, the plasma is removed. In some embodiments, the blood is allowed to clot before centrifugation. In related embodiments, after centrifugation, the serum is removed.

### Measuring Biomarkers

A sample or processed sample may be assayed to determine the amount or concentration of particular biomarkers in the sample. Assays may be performed to measure biomarkers as described below.

In some embodiments biomarkers are measured by using Enzyme-Linked Immunosorbent Assay (ELISA), liquid chromatography mass spectrometry (LCMS), surface enhanced laser desorption ionization time, surface enhanced laser desorption ionization time of flight mass spectrometry (SELDI-TOF), two-dimension gel electrophoresis (2-DE), and or any combinations thereof.

In some embodiments biomarkers are measured using bead-based multiplex immune assays. In some embodiments the bead-based multiplex immune assays further comprise combining Luminex technology with automated liquid handling. In some embodiments the methods includes detection of blood proteins. In some embodiments the methods include using small sample volumes. In some embodiments the sample volume is 10 ul. In some embodiments the sample volume is 20 ul. In some embodiments the sample volume is 30 ul. In some embodiments the sample volume is 40 ul. In some embodiments the sample volume is 50 ul. In some embodiments the sample volume is 60 ul. In some embodiments the sample volume is 70 ul. In some embodiments the sample volume is 80 ul. In some embodiments the sample volume is 10 ul. In some embodiments the sample volume is 90 ul. In some embodiments the sample volume is 100 ul. In some embodiments the methods include measuring biomarkers in a dynamic range of fg/ml to mg/ml.

In some embodiments biomarkers are measured using a multiplexed assay. In some embodiments the multiplexed assay further comprises a microsphere-based assay. In some embodiments multiplex assays are performed in a single reaction vessel. In some embodiments multiplex assays are performed by combining optical classification schemes, biochemical assays, flow cytometry, and advanced digital signal processing hardware and software. In some embodiments as many as 100 multiplex assays are performed in a single reaction vessel. In some embodiments between 1 and 2 multiplex assays are performed in a single reaction vessel. In some embodiments between 1 and 3 multiplex assays are performed in a single reaction vessel. In some embodiments between 1 and 4 multiplex assays are performed in a single reaction vessel. In some embodiments between 1 and 5 multiplex assays are performed in a single reaction vessel. In some embodiments between 1 and 6 multiplex assays are performed in a single reaction vessel. In some embodiments between 1 and 7 multiplex assays are performed in a single reaction vessel. In some embodiments between 1 and 8 multiplex assays are performed in a single reaction vessel. In some embodiments between 1 and 9 multiplex assays are performed in a single reaction vessel. In some embodiments between 1 and 10 multiplex assays are performed in a single reaction vessel. In some embodiments between 1 and 11 multiplex assays are performed in a single reaction vessel. In some embodiments between 1 and 12 multiplex assays are performed in a single reaction vessel. In some embodiments between 1 and 13 multiplex assays are performed in a single reaction vessel. In some embodiments between 1 and 14 multiplex assays are performed in a single reaction vessel. In some embodiments between 1 and 15 multiplex assays are performed in a single reaction vessel. In some embodiments between 1 and 20 multiplex assays are performed in a single reaction vessel. In some embodiments between 1 and 30 multiplex assays are performed in a single reaction vessel. In some embodiments between 1 and 40 multiplex assays are performed in a single reaction vessel. In some embodiments between 1 and 50 multiplex assays are performed in a single reaction vessel. In some embodiments between 1 and 60 multiplex assays are performed in a single reaction vessel. In some embodiments between 1 and 70 multiplex assays are performed in a single reaction vessel. In some embodiments between 1 and 80 multiplex assays are performed in a single reaction vessel. In some embodiments between 1 and 90 multiplex assays are performed in a single reaction vessel. In some embodiments between 1 and 100 multiplex assays are performed in a single reaction vessel.

In some embodiments the multiplex assay includes analyte-specific microspheres labeled with a unique fluorescent signature. In some embodiments microspheres are labeled with red fluorescent dye. In some embodiments microspheres are labeled with far red fluorescent dye. In some embodiments microspheres are labeled with varying intensity of dye.

In some embodiments the multiplex assay includes an assay specific capture reagent that is covalently conjugated to a set of unique microspheres. In some embodiments conjugation of the capture reagent occurs with carbodimide chemistry. In some embodiments conjugation occurs with carboxyl functional groups on the surface of the microspheres. In some embodiments conjugation occurs with primary amines in the capture agent. In some embodiments the assay specific capture reagent is an antibody. In some embodiments the assay specific capture reagent is an antigen, receptor, peptide or enzyme substrate. In some embodiments the assay-specific capture reagent on each individual microsphere binds an analyte of interest.

In some embodiments a detecting reagent is added to the assay. In some embodiments the detecting reagent is biotinylated. In some embodiments the detecting reagent is an antibody, antigen or ligand. In some embodiments the detecting reagent is assay-specific. In some embodiments the detecting reagent is a streptavidin-labelled fluorescent molecule. In some related embodiments, the streptavidin-labelled fluorescent molecule is phycoerythrin.

In some embodiments the assay kinetics are near solution-phase. In some embodiments the multiplex assay is washed to remove unbound detecting reagents. In some embodiments microsphere aggregates are removed from analysis.

In some embodiments the multiplex reaction is passed through an analyzer comprising laser beams. In some embodiments the analyzer uses hydrodynamic focusing to pass microspheres through the analyzer in single file. In some embodiments the individual microspheres pass through at least one excitation beams.

In some embodiments microsphere size is measured. In some embodiments microsphere size is determined by measuring 90-degree light scatter. In some embodiments microsphere size is determined by passing the microspheres through a red laser. In a related embodiment, the red diode laser is a 633 nm diode laser.

In some embodiments the microsphere comprises an encoded fluorescent signature. In some embodiments the encoded fluorescent signature of the microsphere is analyzed. In some embodiments the fluorescent signature is determined by passing the microspheres through a red diode laser. In some embodiments the fluorescent signature is measured using avalanche photodiodes.

In some embodiments microsphere fluorescence is measured. In some embodiments microsphere fluorescence is measured by passing the microspheres through a green laser. In a related embodiment, the green laser is a 532 nm diode laser.

In some embodiments a fluorescent reporter signal is generated in proportion to the analyte concentration. In some embodiments the fluorescence is measured using a photomultiplier tube. In some embodiments at least 100 microspheres from each analyte set are analyzed. In some embodiments the median value of the analyte-specific fluorescence is measured. In some embodiments analyte fluorescence is calibrated using internal controls of known quantity.

In some embodiments, two or more multiplex reactions are processed in parallel. In a related embodiment between 2 and 3, 2 and 4, 2 and 5, 2 and 10, 2 and 20, 2 and 30, 2 and 50, 2 and 100, 2 and 200 or 2 and 400 multiplex reactions are processed in parallel. In a related embodiment, two or more multiplex reactions are processed together in a plate. In related embodiments, multiplex reactions are processed together on a 6, 12, 24, 48, 96 or 384 well plate. In a related embodiment, internal control reactions are run in parallel with multiplex reactions on a plate.

### Models for Distinguishing Mood Disorders

Another aspect of the present disclosure provide models, methods of generating models and methods of using models to distinguish mood disorders.

In one embodiment, the model is a multivariate model. In some embodiments the multivariate model comprises biomarkers. In some embodiments, the multivariate model generated using the panels of biomarkers and covariates disclosed. In some embodiments, the model comprises one or more continuous variables. In some embodiments, the model comprises one or more categorical variables. In some embodiments, the model comprises both continuous and categorical variables.

In some embodiments, the variables in the multivariate model comprise the amount of one or more biomarkers in a biological fluid sample. In some embodiments, the variable in the multivariate model comprise the concentration of one or more biomarkers in a biological fluid sample. In some embodiments, the amounts of the one or more biomarkers in the biological fluid sample are binned to generate categorical variables representing the amount of biomarker. In some embodiments, the concentrations of the one or more biomarkers in the biological fluid sample are binned to generate categorical variables representing the amount of biomarker.

In some embodiments, the variables in the multivariate model comprise the likelihood that an individual suffers from Major Depressive Disorder. In some embodiments, the variables in the multivariate model comprise the likelihood that an individual suffers from a bipolar disorder. In some embodiments, the variables in the multivariate model comprise the likelihood that an individual suffers from Bipolar Disorder I. In some embodiments, the variables in the multivariate model comprise the likelihood that an individual suffers from Bipolar Disorder II. In some embodiments, a cutoff likelihood value is used to express disease state as a categorical value.

Multivariate models useful for the present disclosure include Multivariate analysis of variance (MANOVA), multivariate regression, principal components analysis (PCA), multivariate logistic regression, factor analysis, canonical correlation analysis, redundancy analysis (RDA), correspondence analysis (CA), canonical (or “constrained”) correspondence analysis (CCA), multidimensional scaling, principal coordinates analysis, discriminant analysis, linear discriminant analysis (LDA), clustering systems, recursive partitioning, artificial neural networks, simultaneous equations models, and vector autoregression.

In some embodiments, the multivariate model is selected from Multivariate Logistic Regression (MLR), Principal Component Analysis (PCA), Factor Analysis, Partial Least Squares Regression (PLS), Neural Network, Linear Discriminant Analysis (LDA), K-Nearest Neighbor (KNN) classification, Cluster Analysis or self-organizing maps, or combinations thereof.

In some embodiments, the multivariate model is Multivariate Logistic Regression (MLR). In some embodiments, the multivariate model is Principal Component Analysis (PCA). In some embodiments, the multivariate model is Factor Analysis. In some embodiments, the multivariate model is Partial Least Squares Regression (PLS). In some embodiments, the multivariate model is Neural Network. In some embodiments, the multivariate model is Linear Discriminant Analysis (LDA). In some embodiments, the multivariate model is K-Nearest Neighbor (KNN) classification. In some embodiments, the multivariate model is Cluster Analysis.

Once a model has been generated and optimized, data obtained by an assay may be input into the model in order to generate a result. In some embodiments, the result of inputting data into the model is a continuous variable. In a related embodiment the continuous variable output is an absolute or relative likelihood that an individual suffers from a particular mood disorder. In some embodiments, the result of inputting data into the model is a categorical variable. In a related embodiment the categorical variable output is designation that the individual likely suffers from MDD. In a related embodiment the categorical variable output is designation that the individual likely suffers from a bipolar disorder. In a related embodiment the categorical variable output is designation that the individual likely suffers from BPD1. In a related embodiment the categorical variable output is designation that the individual likely suffers from BPD2

### Treatment of Depression

Health practitioners treat depression by taking actions to ameliorate the causes or symptoms of the disorder in a patient. Treatment may comprise drug-based or non-drug-based therapies.

Drug-based therapies may include: selecting and administering one or more antidepressant drugs to the patient, adjusting the dosage of an antidepressant drug, adjusting the dosing schedule of an antidepressant drug, and adjusting the length of the therapy with an antidepressant drug. Antidepressant drugs are selected by practitioners based on the nature of the symptoms and the patient's response to any previous treatments. The dosage of an antidepressant drug can be adjusted as well by the practitioner based on the nature of the drug, the nature of the patient's symptoms, the patient's response to previous treatment, and the patient's response to the drug. The dosing schedule can also be adjusted by the practitioner based on the nature of the drug, the nature of the patient's symptoms, the patient's response to previous treatment, and the patient's response to the drug. Also, the length of the therapy can be adjusted by the practitioner based on the nature of the drug, the nature of the patient's symptoms, the patient's response to previous treatment, and the patient's response to the drug. Additionally, the practitioner can select between a single drug therapy, a dual drug therapy, or a triple drug therapy. In some embodiments, a practitioner may optionally treat the patient with a combination of one or more antidepressant drugs and one or more non-drug-based therapies.

In some embodiments, treatment comprises administering to an individual a selective serotonin reuptake inhibitor (“SSRI”). In some embodiments, the SSRI is citalopram. In some embodiments, the SSRI is escitalopram. In some embodiments, the SSRI is fluoxetine. In some embodiments, the SSRI is paroxetine. In some embodiments, the SSRI is sertraline.

In other embodiments, treatment comprises administering to an individual a serotonin-norepinephrine reuptake inhibitors (“SNRI”). In some embodiments, the SNRI is venlafaxine. In other embodiments, the SNRI is duloxetine.

In other embodiments, treatment comprises administering to an individual a norepinephrine and dopamine reuptake inhibitor (“NDRI”). In one embodiment, the NDRI is bupropion.

In other embodiments, treatment comprises administering to an individual a tetracyclic antidepressant (“tetracyclic”). In some embodiments, the tetracyclic is amoxapine. In some embodiments, the tetracyclic is maprotiline. In some embodiments, the tetracyclic is mazindol. In some embodiments, the tetracyclic is mirtazapine.

In other embodiments, treatment comprises administering to an individual a tricyclic antidepressant (“tricyclic”). In some embodiments, the tricyclic is amitriptyline. In some embodiments, the tricyclic is imipramine. In some embodiments, the tricyclic is nortriptyline.

In other embodiments, treatment comprises administering to an individual a monoamine oxidase inhibitor (“MAOI”). In some embodiments, the MAOI is selegiline. In some embodiments, the MAOI is isocarboxazid. In some embodiments, the MAOI is phenelzine. In some embodiments, the MAOI is tranylcypromine.

In addition to or in lieu of drug-based therapies, in some embodiments a practitioner may also treat an individual with non-drug-based antidepressant therapies. In some embodiments, the non-drug based therapy comprises cognitive-behavioral therapy. In some embodiments, the non-drug based therapy comprises psychotherapy. In a related embodiment, the non-drug based therapy comprises psychodynamic therapy. In some embodiments, the non-drug based therapy comprises electroconvulsive therapy. In some embodiments, the non-drug based therapy comprises hospitalization and residential treatment programs. In some embodiments, the non-drug based therapy comprises vagus nerve stimulation. In some embodiments, the non-drug based therapy comprises transcranial magnetic stimulation. In some embodiments, the non-drug based therapy comprises regular, vigorous exercise.

### Treatment of Bipolar I and II

Health practitioners treat bipolar disorder I and II by taking actions to ameliorate the causes or symptoms of the disorder in a patient. Treatment may comprise drug-based or non-drug-based therapies.

It is of particular importance that individuals with BPD1 and BPD2 are not treated with antidepressants typically used to treat depression (e.g. SSRIs, etc.), as this can exacerbate manic or hypomanic episodes.

Drug-based therapies may include: selecting and administering one or more drugs to the patient, adjusting the dosage of a drug, adjusting the dosing schedule of a drug, and adjusting the length of the therapy with a drug. Drugs are selected by practitioners based on the nature of the symptoms and the patient's response to any previous treatments. The dosage of a drug can be adjusted as well by the practitioner based on the nature of the drug, the nature of the patient's symptoms, the patient's response to previous treatment, and the patient's response to the drug. The dosing schedule can also be adjusted by the practitioner based on the nature of the drug, the nature of the patient's symptoms, the patient's response to previous treatment, and the patient's response to the drug. Also, the length of the therapy can be adjusted by the practitioner based on the nature of the drug, the nature of the patient's symptoms, the patient's response to previous treatment, and the patient's response to the drug. Additionally, the practitioner can select between a single drug therapy, a dual drug therapy, or a triple drug therapy. In some embodiments, a practitioner may optionally treat the patient with a combination of one or more drugs and one or more non-drug-based therapies.

In some embodiments, treatment comprises administering to an individual lithium.

In other embodiments, treatment comprises administering to an individual an anti-convulsant. In some embodiments, the anti-convulsant is valproate. In some embodiments, the anti-convulsant is lamotrigine. In some embodiments, the anti-convulsant is carbamazepine.

In other embodiments, treatment comprises administering to an individual a traditional antipsychotic. In one embodiment, the traditional antipsychotic is haloperidol. In one embodiment, the traditional antipsychotic is loxapine. In one embodiment, the traditional antipsychotic is chlorpormazine.

In other embodiments, treatment comprises administering to an individual an atypical antipsychotic. In some embodiments, the atypical antipsychotic is aripiprazole. In some embodiments, the atypical antipsychotic is risperidone. In some embodiments, the atypical antipsychotic is asenapine. In some embodiments, the atypical antipsychotic is quentapine. In some embodiments, the atypical antipsychotic is ziprasidone. In some embodiments, the atypical antipsychotic is olanzapine. In some embodiments, the atypical antipsychotic is lurasidone.

In other embodiments, treatment comprises administering to an individual a benzodiazepine. In some embodiments, the benzodiazepine is alprazolam. In some embodiments, the benzodiazepine is lorazepam. In some embodiments, the benzodiazepine is diazepam.

In addition to or in lieu of drug-based therapies, in some embodiments a practitioner may also treat an individual with non-drug-based therapies. In some embodiments, the non-drug based therapy comprises cognitive-behavioral therapy. In some embodiments, the non-drug based therapy comprises psychotherapy. In a related embodiment, the non-drug based therapy comprises psychodynamic therapy. In some embodiments, the non-drug based therapy comprises electroconvulsive therapy. In some embodiments, the non-drug based therapy comprises avoiding stressful situations. In some embodiments, the non-drug based therapy comprises getting adequate rest.

### Methods of Altering Treatment

One aspect of the present disclosure is to provide for methods of altering treatment.

In one embodiment, the method of altering treatment comprises:

a. assaying a body fluid sample, from an individual presenting with depressive symptoms, to determine the amount in the sample of each of a panel of biomarkers;

b. determining, by a model comprising the amounts of each of the panel of biomarkers, that the individual likely has a mood disorder other than Major Depressive Disorder (MDD); and

c. altering the treatment of the individual from antidepressant therapy to treatment of the bipolar disorder.

In related embodiments the individual is being treated with antidepressant therapy before the step of altering the treatment of the individual. In related embodiments the individual is has been treated with antidepressant therapy for up to one week before the step of altering the treatment of the individual. In related embodiments the individual is has been treated with antidepressant therapy for up to two weeks before the step of altering the treatment of the individual. In related embodiments the individual is has been treated with antidepressant therapy for up to three weeks before the step of altering the treatment of the individual. In some embodiments, an attending healthcare provider has intent to treat the individual with antidepressant therapy before the step of altering the treatment of the individual.

In some embodiments, treatment is altered before exacerbation or inducement of mania or hypomania in an individual.

In related embodiments, the antidepressant therapy comprises treating the individual with a drug-based or non-drug-based antidepressant therapy, or both. In some embodiments the drug-based antidepressant therapy comprises a drug selected from a selective serotonin reuptake inhibitor (SSRI), serotonin-norepinephrine reuptake inhibitors (SNRI), norepinephrine and dopamine reuptake inhibitor (NDRI), tetracyclic antidepressant (tetracyclic), tricyclic antidepressant (tricyclic) or monoamine oxidase inhibitor (MAOI), or combinations thereof.

In some embodiments the non-drug-based antidepressant therapy comprises a treatment selected from cognitive-behavioral therapy, psychotherapy, psychodynamic therapy, electroconvulsive therapy, hospitalization and residential treatment programs, vagus nerve stimulation, transcranial magnetic stimulation or regular, vigorous exercise or combinations thereof.

In some embodiments the bipolar disorder is Bipolar Disorder I (BPD1).

In some embodiments the bipolar disorder is Bipolar Disorder II (BPD2).

In some embodiments, altering the treatment of the individual from antidepressant therapy to treatment of the bipolar disorder comprises treatment of BPD1. In some embodiments, altering the treatment of the individual from antidepressant therapy to treatment of the bipolar disorder comprises treatment of BPD2.

In some embodiments, altering the treatment of the individual comprises treating the individual with lithium.

In some embodiments, altering the treatment of the individual comprises treating the individual with an anticonvulsant. In some embodiments, the anticonvulsant is valproate, lamotrigine or carbamazepine.

In some embodiments, altering the treatment of the individual comprises treating the individual with a traditional antipsychotic. In some embodiments, the traditional antipsychotic is haloperidol, loxapine or chlorpromazine.

In some embodiments, altering the treatment of the individual comprises treating the individual with an atypical antipsychotic. In some embodiments, wherein the atypical antipsychotic is aripiprazole, risperidone, asenapine, quetiapine, ziprasidone, olanzapine or lurasidone.

In some embodiments, altering the treatment of the individual comprises treating the individual with a benzodiazepine. In some embodiments, the benzodiazepine is alprazolam, lorazepam or diazepam.

In some embodiments, altering the treatment of the individual comprises treating electroconvulsive therapy (ECT).

In any of the methods of altering treatment, the model may be generated as described herein using the panels of biomarkers and covariates disclosed.

### Methods of Treatment

Another aspect of the present disclosure is to provide for methods of altering treatment.

In one embodiment, the method of treatment comprises:

a. assaying a body fluid sample, from an individual presenting with depressive symptoms, to determine the amount in the sample of each of a panel of biomarkers;

b. determining, by a model comprising the amounts of each of the panel of biomarkers, that the individual likely has a mood disorder other than Major Depressive Disorder (MDD); and

c. administering a treatment to the individual which alleviates the symptoms of the bipolar disorder.

In some embodiments the bipolar disorder is Bipolar Disorder I (BPD1).

In some embodiments the bipolar disorder is Bipolar Disorder II (BPD2).

In some embodiments, administering a treatment to the individual which alleviates the symptoms of the bipolar disorder comprises treatment of BPD1. In some embodiments, administering a treatment to the individual which alleviates the symptoms of the bipolar disorder comprises treatment of BPD2.

In some embodiments, administering treatment to the individual comprises treating the individual with lithium.

In some embodiments, administering treatment to the individual comprises treating the individual with an anticonvulsant. In some embodiments, the anticonvulsant is valproate, lamotrigine or carbamazepine.

In some embodiments, administering treatment to the individual comprises treating the individual with a traditional antipsychotic. In some embodiments, the traditional antipsychotic is haloperidol, loxapine or chlorpromazine.

In some embodiments, administering treatment to the individual comprises treating the individual with an atypical antipsychotic. In some embodiments, wherein the atypical antipsychotic is aripiprazole, risperidone, asenapine, quetiapine, ziprasidone, olanzapine or lurasidone.

In some embodiments, administering treatment to the individual comprises treating the individual with a benzodiazepine. In some embodiments, the benzodiazepine is alprazolam, lorazepam or diazepam.

In some embodiments, administering treatment to the individual comprises treating electroconvulsive therapy (ECT).

In any of the methods treatment, the model may be generated as described herein using the panels of biomarkers and covariates disclosed.

### Methods for Generating Quantitative Data

Another aspect of the present disclosure is to provide methods for generating quantitative data for a subject.

In one embodiment, the method for generating quantitative data for the subject comprises: performing at least one immunoassay on a body fluid sample from the subject to generate a dataset comprising the quantitative data, wherein the quantitative data represents a panel of protein biomarkers, and wherein the individual has a mood disorder other than Major Depressive Disorder (MDD).

In a related embodiment, performance of the at least one immunoassay comprises:

a. obtaining the body fluid sample, wherein the body fluid sample comprises the panel of protein biomarkers;

b. contacting the first sample with a plurality of distinct reagents;

c. generating a plurality of distinct complexes between the reagents and markers; and

d. detecting the complexes to generate the data.

In some embodiments the bipolar disorder is Bipolar Disorder I (BPD1).

In some embodiments the bipolar disorder is Bipolar Disorder II (BPD2).

In any of the methods generating quantitative data, the panel of protein biomarkers may comprise any of the panels of biomarkers and covariates disclosed herein.

### Methods of Differential Diagnosis

Another aspect of the present disclosure is to provide methods of differential diagnosis.

In one embodiment, the method of differential diagnosis comprises:

a. obtaining a body fluid sample from an individual presenting with depressive symptoms;

b. assaying the body fluid sample to determine the amount in the sample of each of a panel of biomarkers;

c. determining, by a model comprising the amounts of each of the panel of biomarkers, that the individual likely has a mood disorder other than Major Depressive Disorder (MDD); and

d. diagnosing the individual as suffering from a bipolar disorder based at least in part on the determining in step b.

In some embodiments, the bipolar disorder is Bipolar Disorder I (BPD1).

In some embodiments, the bipolar disorder is Bipolar Disorder II (BPD2).

In some embodiments, diagnosing the individual as suffering from a bipolar disorder based at least in part on the determining in step b comprises combining the likelihood the individual has a mood disorder other than Major Depressive Disorder (MDD) with one or more clinical factors. In some embodiments, the one or more clinical factors comprise genetic information. In some embodiments, the one or more clinical factors comprise brain imaging. In a related embodiment, the brain imaging is generated suing fMRI or PET.

In some embodiments, the one or more clinical factors comprise the presence of depressive symptoms. In some embodiments, the depressive symptoms comprise an overly long period of feeling sad or hopeless, loss of interest in activities once enjoyed, feeling overly tired or sluggish, having problems concentrating, remembering, and making decisions, being restless or irritable, changing eating, sleeping, or other habits, thinking of death or suicide, or attempting suicide.

In some embodiments, the one or more clinical factors comprise the presence of manic symptoms. In some embodiments, the one or more clinical factors comprise the presence of hypomanic symptoms. In some embodiments, the manic or hypomanic symptoms may comprise an overly long period of feeling “high,” or an overly happy or outgoing mood, extreme irritability, talking very fast, jumping from one idea to another, having racing thoughts, being unusually distracted increasing activities, such as taking on multiple new projects, being overly restless, sleeping little or not being tired, having an unrealistic belief in your abilities, behaving impulsively and engaging in pleasurable, high-risk behaviors, or any combination thereof.

In some embodiments, the one or more clinical factors comprise the presence of mood cycling. In a related embodiment, the one or more clinical factors comprise the rate of mood cycling.

In any of the methods treatment, the model may be generated as described herein using the panels of biomarkers and covariates disclosed.

### Methods of Mitigating Mania or Hypomania

Another aspect of the present disclosure is to provide methods of differential diagnosis.

In one embodiment, the method of mitigating antidepressant-exacerbated mania or hypomania comprises:

a. assaying a body fluid sample, from an individual presenting with depressive symptoms, to determine the amount in the sample of each of a panel of biomarkers;

b. determining, by a model comprising the amounts of each of the panel of biomarkers, that the individual likely has a mood disorder other than Major Depressive Disorder (MDD); and

c. altering the treatment of the individual by: not beginning treatment with antidepressant therapy or discontinuing treatment with antidepressant therapy.

In some embodiments, the individual is being treated with antidepressant therapy before the step of altering the treatment of the individual.

In some embodiments, the antidepressant therapy comprises treating the individual with a drug-based therapy.

In some embodiments, the drug-based antidepressant therapy comprises a drug selected from a selective serotonin reuptake inhibitor (SSRI), serotonin-norepinephrine reuptake inhibitors (SNRI), norepinephrine and dopamine reuptake inhibitor (NDRI), tetracyclic antidepressant (tetracyclic), tricyclic antidepressant (tricyclic) or monoamine oxidase inhibitor (MAOI), or combinations thereof.

In some embodiments, the mood disorder other than MDD is Bipolar Disorder I (BPD1).

In some embodiments, the mood disorder other than MDD is Bipolar Disorder II (BPD2).

In some embodiments, altering the treatment of the individual comprises treating the individual with lithium.

In some embodiments, altering the treatment of the individual comprises treating the individual with an anticonvulsant. In a related embodiments, the anticonvulsant is valproate, lamotrigine or carbamazepine.

In some embodiments, altering the treatment of the individual comprises treating the individual with a traditional antipsychotic. In a related embodiment the traditional antipsychotic is haloperidol, loxapine or chlorpromazine.

In some embodiments, altering the treatment of the individual comprises treating the individual with an atypical antipsychotic. In a related embodiment, the atypical antipsychotic is aripiprazole, risperidone, asenapine, quetiapine, ziprasidone, olanzapine or lurasidone.

In some embodiments, altering the treatment of the individual comprises treating the individual with a benzodiazepine. In a related embodiment the benzodiazepine is alprazolam, lorazepam or diazepam.

In some embodiments, altering the treatment of the individual comprises treating electroconvulsive therapy (ECT).

In any of the methods treatment, the model may be generated as described herein using the panels of biomarkers and covariates disclosed.

The following examples further elucidate many aspects of the present disclosure.

### EXAMPLES

### Example 1

This example describes the discovery of biomarkers that distinguish bipolar disorder (BPD) from major depressive disorder (MDD).

A study was conducted that enrolled 147 samples. The distribution by disease status was 46 Bipolar disorder type 1 (BPD1), 49 Bipolar Disorder type II (BPD2), and 52 MDD. Disease classification, age, gender, and body mass index were used as clinical variables.

Samples underwent proteomic analyte testing on Myriad RBM's Human DiscoveryMap 250+ platform with 320 analytes measured. The samples were randomized across disease states over 4 plates of 72 samples each. Log 10 transformation was applied to 294 proteomic analyte measurements to stabilize variance patters. Proteomic analyte measurements lower than a lower limit of quantitation (LLOQ) were replaced by averages of the least detectable does (LDD) and LLOQ. Forty-three of 277 proteomic analytes were dropped from analyses for having constant values for all observations (17 analytes) or near constant values, i.e., >95% values (26 analytes).

Correlation analyses were conducted for the remaining group of 277 analytes. Of a total of 38,226 pairwise combinations, 87 (0.2%) analyte pairs were correlated with an absolute value of correlation |r|>0.6.

Univariate analyses were conducted to correlate baseline proteomic analytes and demographic variables (age, gender, and BMI) to disease classification were conducted. BMI and gender did not show significant differences with respect to disease classification. Inclusion of age also did not add meaningful value to the predictive power of the developed models. Univariate analyses (ANOVA) was conducted on the 277 analytes. A set of 74 analytes were identified. Density plots were created for each of the 74 analytes to help visualize the relative distribution of each analyte over the 3 disease groups.

Multivariate analyses were then conducted employing linear discriminant analysis (LDA) and multivariate logistic regression, which resulted in 30 analytes useful for distinguishing the 3 disease groups (Table 1).

TABLE 1
Analytes that distinguish BPD1, BPD2, and MDD.

RBP4
LGL
EGF
MMP7
TIE2
Insulin
GDF15
ITAC
MIP1beta
Aldose Reductase
Omentin
TIMP2
TNFR2
Progesterone
PARC
NrCAM
TAFI
IGFBP1
Galectin3
C3
CD40L
TSH
IGFBP4
Hemopexin
VEGF
PEDF
TTR
MMP2
B2M
SAP

From the final list of 30 analytes, four models were developed to distinguish the 3 disease groups: 1) MDD versus BPD1/2 (i.e., BPD1 and BPD2 combined); 2) BPD1 versus MDD; 3) BPD1 versus BPD2, and 4) BPD2 versus MDD. Each LDA model was developed in two steps using the PROC DISCRIM procedure in SAS System (V9.2). First, a stepwise selection method was used over the analyte set to identify the best subset of analytes to classify the two disease groups being compared for each respective model. More relaxed cut-offs for entry (p-value <0.3) and exit (p-value <0.1) were used as a first step. Next, the best LDA model was fitted and its training and cross-validation performance was measured. Table 2 provides a list of biomarkers that can be used to distinguish BPD1/2 from MDD. An LDA model generated using the markers in Table 2 provides a training AUROC of 0.82 and a cross-validation AUROC of 0.75.

TABLE 2
Biomarkers that distinguish BPD1/2 from MDD

EGF
TIE2
Insulin
GDF15
ITAC
MIP1beta
Aldose Reductase
TIMP2
TNFR2

Table 3 provides a list of biomarkers that can be used to distinguish BPD1 from BPD2. An LDA model generated using the markers in Table 3 provides a training AUROC of 0.84 and a cross-validation AUROC of 0.80.

TABLE 3
Biomarkers that distinguish BPD1 from BPD2

RBP4
Omentin
Progesterone
PARC
NrCAM
TAFI
IFGBP1

Table 4 provides a list of biomarkers that can be used to distinguish BPD1 from BPD2, which includes covariate analytes. Each of the covariate analytes can be used as auxiliary analytes to replace or complement the primary analyte to its left in the table.

TABLE 4
Biomarkers that distinguish BPD1 from BPD2

| Primary analytes | Covariate analytes |
|---|---|
| RBP4 | Hemopexin, TTR |
| Omentin | |
| Progesterone | |
| PARC | |
| NrCAM | |
| TAFI | C3 |
| IFGBP1 | |

Table 5 provides a list of biomarkers that can be used to distinguish BPD1 from BPD2 from MDD. The markers in Table 5 can be used to distinguish BPD1 from BPD2; BPD1 from MDD; and BPD2 from MDD.

TABLE 5
Biomarkers that distinguish BPD1 from BPD2 from MDD

RBP4
LGL
EGF
MMP7
TIE2
Insulin
MIP1beta
Aldose Reductase
TIMP2
Progesterone
PARC
Galectin3
C3

Table 6 provides a list of biomarkers that can be used to distinguish BPD1 from BPD2 from MDD. The markers in Table 6 can be used to distinguish BPD1 from BPD2; BPD1 from MDD; and BPD2 from MDD. The markers in Table 6 include covariate analytes. Each of the covariate analytes can be used as auxiliary analytes to replace or complement the primary analytes.

TABLE 6
Biomarkers that distinguish BPD1 from BPD2 from MDD

Primary analytes
RBP4
LGL
EGF
MMP7
TIE2
Insulin
MIP1beta
Aldose Reductase
TIMP2
Progesterone
PARC
Galectin3
C3
Covariate analytes
Hemopexin
TTR
MMP 2
SAP

Table 7 provides a list of biomarkers that can be used to distinguish BPD2 from MDD. An LDA model generated using the markers in Table 7 provides a training AUROC of 0.80 and a cross-validation AUROC of 0.77.

TABLE 7
Biomarkers that distinguish BPD2 from MDD

GDF15
TIMP2
CD40L
TSH
IGFBP4
Hemopexin
VEGF

Table 8 provides a list of biomarkers that can be used to distinguish BPD2 from MDD, which includes covariate analytes. Each of the covariate analytes can be used as auxiliary analytes to replace or complement the primary analyte to its left in the table.

TABLE 8
Biomarkers that distinguish BPD2 from MDD

| Primary analytes | Covariate analytes |
|---|---|
| GDF15 | |
| TIMP2 | MMP 2 |
| CD40L | |
| TSH | |
| IGFBP4 | |
| Hemopexin | RBP4, C3 |
| VEGF | |

Table 9 provides a list of biomarkers that can be used to distinguish BPD1 from MDD. An LDA model generated using the markers in Table 9 provides a training AUROC of 0.96 and a cross-validation AUROC of 0.92.

TABLE 9
Biomarkers that distinguish BPD1 from MDD

RBP 4
LGL
EGF
PARC
Progesterone
TIE 2
Galectin 3
C3
Aldose Reductase
MIP 1 beta
MMP 7
Insulin
TIMP 2

Table 10 provides a list of biomarkers that can be used to distinguish BPD1 from MDD, which includes covariate analytes. Each of the covariate analytes can be used as auxiliary analytes to replace or complement the primary analyte to its left in the table.

TABLE 10
Biomarkers that distinguish BPD1 from MDD

| Primary analytes | Covariate analytes |
|---|---|
| RBP 4 | TTR |
| LGL | |
| EGF | |
| PARC | |
| Progesterone | |
| TIE 2 | |
| Galectin 3 | |
| C3 | Hemopexin, SAP |
| Aldose Reductase | |
| MIP 1cbeta | |
| MMP 7 | |
| Insulin | |
| TIMP 2 | MMP 2 |

### Example 2

The analyses from Example 1 were performed again, but multivariate logistic regression (MLR) was used to build models and identify analytes.

Table 11 provides a list of biomarkers that can be used to distinguish BPD1/2 from MDD based on the MLR analysis. An MLR model generated using the markers in Table 11 provides a training AUROC of 0.92 and a cross-validation AUROC of 0.88.

TABLE 11
Biomarkers that distinguish BPD1/2 from MDD

EGF
TIMP 2
GDF 15
TIE 2
Insulin
Aldose Reductase
FAS
MIP 1 beta
ITAC
Galectin 3

Table 12 provides a list of biomarkers that can be used to distinguish BPD1 from BPD2. An MLR model generated using the markers in Table 12 provides a training AUROC of 0.93 and a cross-validation AUROC of 0.88.

TABLE 12
Biomarkers that distinguish BPD1 from BPD2

RBP4
Progesterone
MMP 7
Insulin
MCP 1
EGFR
LGL
AB 40

Table 13 provides a list of biomarkers that can be used to distinguish BPD2 from MDD. An MLR model generated using the markers in Table 13 provides a training AUROC of 0.83 and a cross-validation AUROC of 0.76.

TABLE 13
Biomarkers that distinguish BPD2 from MDD

CD40 L
GDF 15
TIMP 2
TSH
Hemopexin
EGFR

Table 14 provides a list of biomarkers that can be used to distinguish BPD1 from MDD. An MLR model generated using the markers in Table 14 provides a training AUROC of 0.98 and a cross-validation AUROC of 0.94.

TABLE 14
Biomarkers that distinguish BPD1 from MDD

RBP4
LGL
EGF
PARC
C3
TIMP 2
TIE 2
MMP 7
Aldose Reductase
IGFBP 1

### Example 3

In this example we attempted to determine, for the set of biomarkers in table 10, key subpanels of biomarkers which comprise core groups which can distinguish BP1 and MDD. based on the set of biomarkers in 10, we picked different combinations of 3 analytes and computed AUC for a model including only the 3 chosen analytes. This gave us a measure of how well a core group of the 3 analytes will classify between the disease states. Next with the remaining set of analytes (except known strong covariates of the chosen 3 analytes) we used a step-wise model selection method to build the best possible model with any number of analytes and compute its AUC. This gives us a measure of how well any other group of analytes will classify between the disease states without the 3 chosen analytes.

Any group of 3 chosen analytes is identified as a core group, if the 3 analytes by themselves provide an AUC exceeding 0.70 in cross-validation, and where the AUC from the best model using remaining analytes (except covariates of the chosen 3 analytes) falls short of an AUROC of 0.80.

Table 15 shows the results for core groups identified and the AUROC for models generated using LDA with the three analytes.

TABLE 15
Core groups and AUROCs based on LDA analysis

| | | | | Cross Validation |
|---|---|---|---|---|
| Core groups | | | Training AUROC | AUROC |
| RBP 4 | EGF | PARC | 0.7653 | 0.7245 |
| RBP 4 | EGF | MMP 7 | 0.7653 | 0.7449 |
| RBP 4 | PARC | MMP 7 | 0.7347 | 0.7143 |
| RBP 4 | MMP 7 | TIMP 2 | 0.7551 | 0.7449 |
| EGF | PARC | MMP 7 | 0.7449 | 0.7245 |
| TTR | EGF | PARC | 0.7653 | 0.7449 |
| TTR | EGF | MMP 7 | 0.7347 | 0.7041 |
| RBP 4 | MMP 7 | MMP 2 | 0.7653 | 0.7449 |

Table 16 shows the AUROC values for the same panels as Table 15, but for models generated using MLR with the three analytes.

TABLE 16
Core groups and AUROCs based on MLR analysis

| | | | | Cross Validation |
|---|---|---|---|---|
| Core Groups | | | Training AUROC | AUROC |
| RBP 4 | EGF | PARC | 0.8361 | 0.8035 |
| RBP 4 | EGF | MMP 7 | 0.8227 | 0.788 |
| RBP 4 | PARC | MMP 7 | 0.7935 | 0.7525 |
| RBP 4 | MMP 7 | TIMP 2 | 0.8018 | 0.7734 |
| EGF | PARC | MMP 7 | 0.801 | 0.7588 |
| TTR | EGF | PARC | 0.7989 | 0.7554 |
| TTR | EGF | MMP 7 | 0.7788 | 0.7333 |
| TTR | PARC | MMP 7 | 0.7467 | 0.7003 |
| TTR | MMP 7 | TIMP 2 | 0.7705 | 0.7278 |
| RBP 4 | MMP 7 | MMP 2 | 0.7876 | 0.7533 |

Other sub-panels which did not meet the strict criteria for “core group,” but which a) are relatively robust, and b) consist of the analytes in a core group, except for the substitution of a covariate as described in Table 10, appear in Table 17, below. Three of the panels are reported with AUROCs generated in LDA analysis, and one with AUROC generated from MLR. Like the core group markers, these markers would together be useful to form a sub-panel core of a larger panel of biomarkers with high clinical sensitivity and specificity.

TABLE 17
Additional Sub-Panels

| | | | | Cross | Model |
|---|---|---|---|---|---|
| | | | Training | Validation | Generated |
| Sub-Panels | | | AUROC | AUROC | From |
| TTR | PARC | MMP 7 | 0.6633 | 0.6531 | LDA |
| TTR | MMP 7 | TIMP 2 | 0.7347 | 0.6837 | LDA |
| TTR | MMP 7 | MMP 2 | 0.7143 | 0.6939 | LDA |
| TTR | MMP 7 | MMP 2 | 0.7496 | 0.6986 | MLR |

1. A method of altering treatment, comprising:
a. assaying a body fluid sample, from an individual presenting with depressive symptoms, to determine the amount in the sample of each of a panel of biomarkers;
b. determining, by a model comprising the amounts of each of the panel of biomarkers, that the individual likely has a mood disorder other than Major Depressive Disorder (MDD); and
c. altering the treatment of the individual from antidepressant therapy to treatment of the bipolar disorder,
wherein the panel of biomarkers comprises one or more of the proteins listed in Table 1, 2, 3, 4, 5, 6, 7, 8, 9, 10, 11, 12, 13 or 14.

2. The method of claim 1, wherein the individual is being treated with antidepressant therapy before the step of altering the treatment of the individual.

3. The method of claim 2, wherein the antidepressant therapy comprises treating the individual with a drug-based or non-drug-based antidepressant therapy, or both.

4. The method of claim 3, wherein the drug-based antidepressant therapy comprises a drug selected from a selective serotonin reuptake inhibitor (SSRI), serotonin-norepinephrine reuptake inhibitors (SNRI), norepinephrine and dopamine reuptake inhibitor (NDRI), tetracyclic antidepressant (tetracyclic), tricyclic antidepressant (tricyclic) or monoamine oxidase inhibitor (MAOI), or combinations thereof.

5. The method of claim 3, wherein the non-drug-based antidepressant therapy comprises a treatment selected from cognitive-behavioral therapy, psychotherapy, psychodynamic therapy, electroconvulsive therapy, hospitalization and residential treatment programs, vagus nerve stimulation, transcranial magnetic stimulation or regular, vigorous exercise or combinations thereof.

6. The method of claim 1, wherein the bipolar disorder is Bipolar Disorder I (BPD1).

7. The method of claim 1, wherein the bipolar disorder is Bipolar Disorder II (BPD2).

8. The method of claim 1, wherein the body fluid sample comprises urine, blood, plasma, serum, buffy coat, or saliva.

9. The method of claim 8, wherein the body fluid sample comprises blood, plasma or serum.

10. The method of claim 1, wherein the model is generated using a statistical method comprising Multivariate Logistic Regression (MLR), Principal Component Analysis (PCA), Factor Analysis, Partial Least Squares Regression (PLS), Neural Network, Linear Discriminant Analysis (LDA), K-Nearest Neighbor (KNN) classification, Cluster Analysis or self-organizing maps, or combinations thereof.

11. The method of claim 10, wherein the model is generated using LDA or MLR.

12. The method of claim 1, wherein assaying the body fluid sample comprises using Western Blot, Enzyme-Linked Immunosorbent Assay (ELISA), liquid chromatography mass spectrometry (LCMS), surface enhanced laser desorption ionization time of flight mass spectrometry (SELDI-TOF), two-dimension gel electrophoresis (2-DE), and/or any combinations thereof.

13. The method of claim 1, wherein assaying the body fluid sample comprises using multiplexed immune assays.

14. (canceled)

15. The method of claim 13, wherein between 1 and 15 multiplexed assays are performed in a single reaction vessel.

16-26. (canceled)

27. The method of claim 1, wherein altering the treatment of the individual comprises treating the individual with lithium.

28. The method of claim 1, wherein altering the treatment of the individual comprises treating the individual with an anticonvulsant.

29. The method of claim 28, wherein the anticonvulsant is valproate, lamotrigine or carbamazepine.

30. The method of claim 1, wherein altering the treatment of the individual comprises treating the individual with a traditional antipsychotic.

31. The method of claim 30, wherein the traditional antipsychotic is haloperidol, loxapine or chlorpromazine.

32. The method of claim 1, wherein altering the treatment of the individual comprises treating the individual with an atypical antipsychotic.

33. The method of claim 32, wherein, wherein the atypical antipsychotic is aripiprazole, risperidone, asenapine, quetiapine, ziprasidone, olanzapine or lurasidone.

34. The method of claim 1, wherein altering the treatment of the individual comprises treating the individual with a benzodiazepine.

35. The method of claim 34, wherein, wherein the benzodiazepine is alprazolam, lorazepam or diazepam.

36. The method of claim 1, wherein altering the treatment of the individual comprises treating electroconvulsive therapy (ECT).

37. (canceled)

38. The method of claim 1, wherein the panel of biomarkers comprises a core group of three proteins listed in Table 15 or 16.

39. The method of claim 1, wherein the panel of biomarkers comprises a sub-panel of three proteins listed in Table 17.

40-159. (canceled)
